(19) United States
(12) Patent Application Publication
(10) Pub. No.: US 2007/0197569 A1
(43) Pub. Date: Aug. 23, 2007

(54) Method for treating central nervous system disorders with substituted 2-imidazoline derivatives

(76) Inventors: Marius Hoener, Basel (CH); Sabine Kolczewski, Rheinfelden (DE); Henri Stalder, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/655,484

(22) Filed: Jan. 19, 2007

(30) Foreign Application Priority Data
Jan. 27, 2006 (EP) 06100953.6

(51) Int. Cl. A61K 31/415; A61K 31/505; C07D 233/44; C07D 239/02

(52) U.S. Cl. 514/269; 514/401; 514/402; 544/319; 548/315.1; 548/331.1

(57) ABSTRACT

The present invention relates to a method for treating depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders which comprises administering to an individual a therapeutically effective amount of a compound of formula I

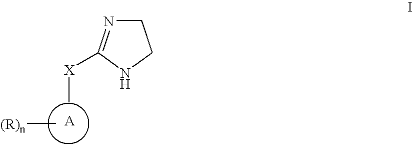

wherein R, X, A, and n are as defined in the specification and pharmaceutically active salts, racemic mixtures, enantiomers, optical isomers and tautomeric forms thereof.

### PRIORITY TO RELATED APPLICATIONS

This application claims the benefit of European Application No. 06100953.6, filed Jan. 27, 2006, which is hereby incorporated by reference in its entirety.

### BACKGROUND OF THE INVENTION

The classical biogenic amines (serotonin, norepinephrine, epinephrine, dopamine, histamine) play important roles as neurotransmitters in the central and peripheral nervous system [1]. Their synthesis and storage, as well as their degradation and reuptake after release are tightly regulated. An imbalance in the levels of biogenic amines is known to be responsible for the altered brain function under many pathological conditions [2-5]. A second class of endogenous amine compounds, the so-called trace amines (TAs) significantly overlap with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines [6].

Their dysregulation has been linked to various psychiatric diseases like schizophrenia and depression [7] and for other conditions like attention deficit hyperactivity disorder, migraine headache, Parkinson's disease, substance abuse and eating disorders [8,9].

For a long time, TA-specific receptors had only been hypothesized based on anatomically discrete high-affinity TA binding sites in the CNS of humans and other mammals [10,11]. Accordingly, the pharmacological effects of TAs were believed to be mediated through the well known machinery of classical biogenic amines, by either triggering their release, inhibiting their reuptake or by “crossreacting” with their receptor systems [9,12,13]. This view changed significantly with the recent identification of several members of a novel family of GPCRs, the trace amine associated receptors (TAARs) [7,14]. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies [7,14]. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR1 ligands have a high potential for the treatment of these diseases.

Therefore, there is a broad interest to increase the knowledge about trace amine associated receptors.

### REFERENCES USED

1 Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press;
2 Wong, M. L. and Licinio, J. (2001) Research and treatment approaches to depression. Nat. Rev. Neurosci. 2, 343-351;
3 Carlsson, A. et al. (2001) Interactions between monoamines, glutamate, and GABA in schizophrenia: new evidence. Annu. Rev. Pharmacol. Toxicol. 41, 237-260;
4 Tuite, P. and Riss, J. (2003) Recent developments in the pharmacological treatment of Parkinson's disease. Expert Opin. Investig. Drugs 12, 1335-1352,
5 Castellanos, F. X. and Tannock, R. (2002) Neuroscience of attention-deficit/hyperactivity disorder: the search for endophenotypes. Nat. Rev. Neurosci. 3, 617-628;
6 Usdin, E. and Sandler, M. eds. (1984), Trace Amines and the brain, Dekker;
7 Lindemann, L. and Hoener, M. (2005) A renaissance in trace amines inspired by a novel GPCR family. Trends in Pharmacol. Sci. 26, 274-281;
8 Branchek, T. A. and Blackburn, T. P. (2003) Trace amine receptors as targets for novel therapeutics: legend, myth and fact. Curr. Opin. Pharmacol. 3, 90-97;
9 Premont, R. T. et al. (2001) Following the trace of elusive amines. Proc. Natl. Acad. Sci. U.S.A. 98, 9474-9475;
10 Mousseau, D. D. and Butterworth, R. F. (1995) A high-affinity [3H] tryptamine binding site in human brain. Prog. Brain Res. 106, 285-291;
11 McCormack, J. K. et al. (1986) Autoradiographic localization of tryptamine binding sites in the rat and dog central nervous system. J. Neurosci. 6, 94-101;
12 Dyck, L. E. (1989) Release of some endogenous trace amines from rat striatal slices in the presence and absence of a monoamine oxidase inhibitor. Life Sci. 44, 1149-1156;
13 Parker, E. M. and Cubeddu, L. X. (1988) Comparative effects of amphetamine, phenylethylamine and related drugs on dopamine efflux, dopamine uptake and mazindol binding. J. Pharmacol. Exp. Ther. 245, 199-210;
14 Lindemann, L. et al. (2005) Trace amine associated receptors form structurally and functionally distinct subfamilies of novel G protein-coupled receptors. Genomics 85, 372-385.

### SUMMARY OF THE INVENTION

The present invention provides a method for treating a disorder selected from depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders by administering to an individual a therapeutically effective amount of compounds of formula I

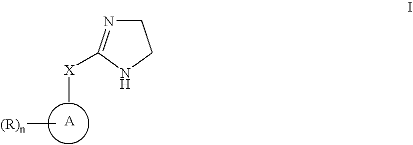

wherein
R is hydrogen, hydroxy, lower alkyl, lower alkoxy, halogen, lower alkyl substituted by halogen, or is 4—(CH₂)₂C(O)-naphthyl;
X is —S— or —NH—;
A is aryl or hetaryl
aryl is an aromatic group selected from the group consisting of phenyl, naphthalen-1-yl, naphthalen-2-yl and 5,6,7,8-tetrahydronaphthalen-1-yl;
hetaryl is an aromatic group containing at least one N or S ring atom selected from the group consisting of thiophen-3-yl and pyrimidin-5-yl; and
n is 1, 2 or 3;
and their pharmaceutically active salts, racemic mixtures, enantiomers, optical isomers and tautomeric forms.

The preferred indications using the compounds of the present invention are depression, psychosis, Parkinson's disease, anxiety and attention deficit hyperactivity disorder (ADHD).

The compounds disclosed in formula I are known compounds, described for example in U.S. Pat. No. 6,268,389 or in the below mentioned references, or are enclosed in public chemical libraries.

Compounds of formula I have a good affinity to the trace amine associated receptors (TAARs), especially for TAAR1.

The present invention also provides pharmaceutical compositions which comprise a therapeutically effective amount of a compound of the invention and a pharmaceutically acceptable carrier.

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, the term “lower alkyl” denotes a saturated straight- or branched-chain hydrocarbon group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkoxy” denotes a group wherein the alkyl residue is as defined above and which is attached via an oxygen atom.

As used herein, the term “lower alkyl substituted by halogen” denotes an alkyl group as defined above, wherein at least one hydrogen atom is replaced by a halogen atom, for example CF₃, CHF₂, CH₂F, CH₂CF₃, CH₂CF₂CF₃ and the like.

The term “halogen” denotes chlorine, iodine, fluorine and bromine.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

The present invention provides a method for treating a disorder selected from depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders by administering to an individual a therapeutically effective amount of compounds of formula I

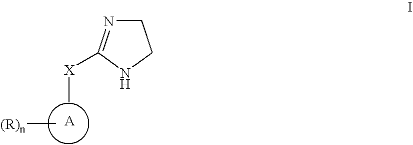

wherein
R is hydrogen, hydroxy, lower alkyl, lower alkoxy, halogen, lower alkyl substituted by halogen, or is 4—(CH₂)₂C(O)-naphthyl;
X is —S— or —NH—;
A is aryl or hetaryl
aryl is an aromatic group selected from the group consisting of phenyl, naphthalen-1-yl, naphthalen-2-yl and 5,6,7,8-tetrahydronaphthalen-1-yl;
hetaryl is an aromatic group containing at least one N or S ring atom selected from the group consisting of thiophen-3-yl and pyrimidin-5-yl; and
n is 1, 2 or 3;
and their pharmaceutically active salts, racemic mixtures, enantiomers, optical isomers and tautomeric forms.

Preferred compounds for use in the methods described above are those, wherein X is N and aryl is phenyl, for example the following compounds
(4,5-dihydro-1H-imidazol-2-yl)-(2,6-dimethyl-phenyl)-amine or tautomer,
(2,6-diethyl-phenyl)-(4,5-dihydro-1H-imidazol-2-yl)-amine or tautomer,
(2,6-dibromo-phenyl)-imidazolidin-2-ylidene-amine or tautomer,
(4,5-dihydro-1H-imidazol-2-yl)-(2-ethyl-6-methyl-phenyl)-amine or tautomer,
(4,5-dihydro-1H-imidazol-2-yl)-(2-isopropyl-6-methyl-phenyl)-amine or tautomer,
(5-chloro-2-methyl-phenyl)-imidazolidin-2-ylidene-amine or tautomer and
3-[4-(4,5-dihydro-1H-imidazol-2-ylamino)-phenyl]-1-naphthalen-2-yl-propan-1-one or tautomer.

Further preferred compounds for use in the method of the invention are those, wherein X is N, and aryl/hetaryl is naphtha-1-yl, 5,6,7,8-tetrahydronaphthalen-1-yl or thiophen-3-yl, for example the following compounds
imidazolidin-2-ylidene-naphthalen-1-yl-amine or tautomer,
(4,5-dihydro-1H-imidazol-2-yl)-(5,6,7,8-tetrahydro-naphthalen-1-yl)-amine or tautomer and
(2-chloro-4-methyl-thiophen-3-yl)-(4,5-dihydro-1H-imidazol-2-yl)-amine or tautomer.

Preferred compounds are further those, wherein X is S and aryl is phenyl, for example 2-(2,6-dichloro-phenylsulfanyl)-4,5-dihydro-1H-imidazole.

The present compounds of formula I and their pharmaceutically acceptable salts can be prepared by methods known in the art, for example, by processes described below, which process comprises
a) reacting a compound of formula

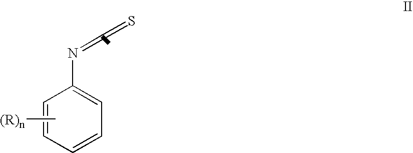

with ethylenediamine of formula

H₂NCH₂CH₂NH₂ III

to obtain a compound of formula

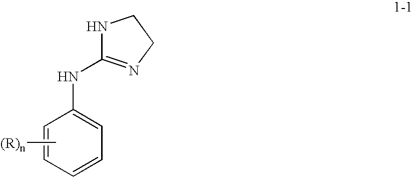

wherein R and n are as defined above, or
b) reacting a compound of formula

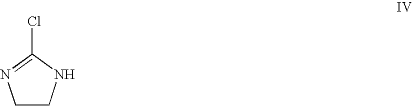

with a compound of formula

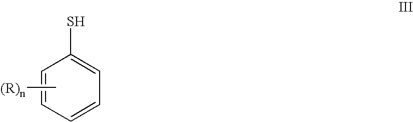

to obtain a compound of formula

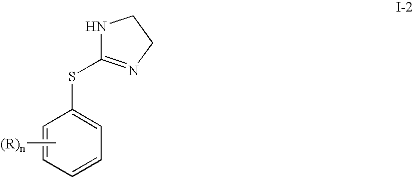

wherein the substituents are as defined above, and

if desired, converting the compounds obtained into pharmaceutically acceptable acid addition salts.

All starting materials are known compounds or can be prepared by processes known in the art.

The 2-aryl/hetaryl-imidazolines were prepared in analogy to literature procedures following the pathway depicted in scheme 1 and scheme 2.
[1] Synthesis 1984, 825
[2] DE 0842065
[3] J. Heterocycl. Chem. 11, 257 (1974)

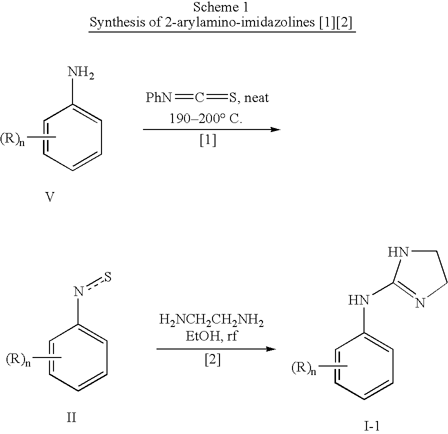

The formation of the imidazoline ring was accomplished by cyclization of an aryl isothiocyanate (II) with ethylenediamine or an analogue thereof in an alcohol, preferred methanol or ethanol, at ambient temperature to reflux temperature, preferred at reflux temperature, for 6 to 48 hours, preferred 18 to 24 hours. The isothiocyanates were prepared from aniline (V) or derivatives thereof by reaction with phenyl isothiocyanate in an inert solvent or neat, preferred neat, at reflux temperature.

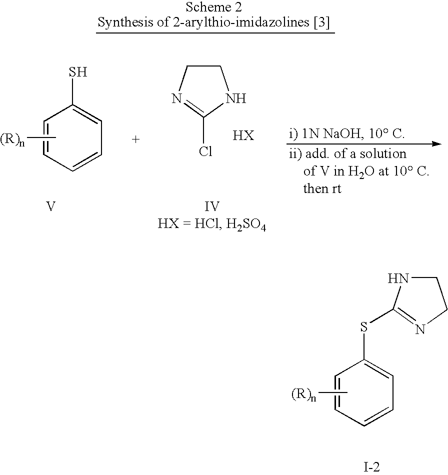

### 2-Aryl/hetaryl-thio-imidazolines can be prepared following a literature procedure depicted in scheme 2.

The compounds mentioned in the table below can be prepared in accordance with the description for Example 2.

### (4,5-Dihydro-1H-imidazol-2-yl)-(2,6-dimethyl-phenyl)-amine or tautomer

### EXAMPLE 2

### a) 2-Isothiocyanato-1,3-dimethyl-benzene

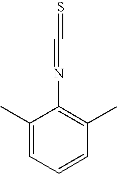

A mixture of 4.00 g (33.0 mmol) 2,6-dimethylaniline and 9.80 g (72.5 mmol) phenyl isothiocyanate was heated to reflux (oil bath 190° C. to 200° C.) for 6 hours. The mixture formed a solid mass when cooled to ambient temperature. To this solid 40 ml n-hexane were added and the suspension stirred for 15 minutes, the precipitate filtered off, washed with n-hexane and the filtrate evaporated. The resulting yellow oil was purified by flash chromatography on silica gel with heptane as eluent and the resulting colourless oil was submitted to a Kugelrohr distillation to get rid of phenyl isocyanate. 2-Isothiocyanato-1,3-dimethyl-benzene was isolated as colourless oil of b.p. 110-120° C./1.2 mbar: MS (EI): 163.1 (M^{+.}).

### b) (4,5-Dihydro-1H-imidazol-2-yl)-(2,6-dimethyl-phenyl)-amine or tautomer

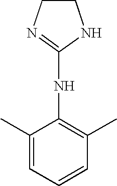

A mixture of 343 mg (806 mmol) sodium hydroxide (crushed pellets) and 0.41 ml (368 mg, 6.1 mmol) ethylenediamine in 6 ml ethanol was stirred at ambient temperature until a solution was obtained. To this solution was added drop-wise a solution of 1.00 g (6.1 mmol) 2-isothiocyanato-1,3-dimethyl-benzene in 2 ml ethanol and the resulting mixture heated to reflux for 20 hours. The resulting yellow solution was cooled to ambient temperature and acidified to pH˜2 by bubbling hydrogen chloride through it. The suspension was filtered, the residue well washed with ethanol and the filtrate evaporated. The residue was dissolved in water, pH adjusted to 10 to 11 and the solution extracted with tert-butyl methyl ether. The combined organic layers were washed with brine, dried over Na₂SO₄ and evaporated. The resulting crude product was purified by flasch-chromatography on silica gel: the impurities were eluted by methanol followed by elution of the title compound with methanol/concentrated ammonia 95:5. (4,5-Dihydro-1H-imidazol-2-yl)-(2,6-dimethyl-phenyl)-amine was isolated as colourless oil which crystallised at ambient temperature: colourless solid, m.p. 155-157° C., MS (ISP): 190.4 (M+H^{+.}).

| Compound | Name | Example |
|---|---|---|
| 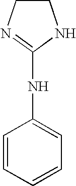 | (4,5-Dihydro-1H-imidazol-2-yl)-phenyl-amineor tautomer | 1 |
| 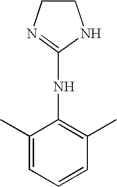 | (4,5-Dihydro-1H-imidazol-2-yl)-(2,6-dimethyl-phenyl)-amine or tautomer | 2 |
| 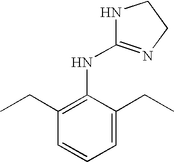 | (2,6-Diethyl-phenyl)-(4,5-dihydro-1H-imidazol-2-yl)-amine or tautomer | 3 |
| 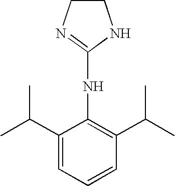 | (4,5-Dihydro-1H-imidazol-2-yl)-(2,6-diisopropyl-phenyl)-amine or tautomer | 4 |
| 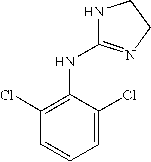 | (2,6-Dichloro-phenyl)-(4,5-dihydro-1H-imidazol-2-yl)-amine or tautomer; Clonidine; | 5 |
| 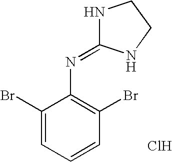 | (2,6-Dibromo-phenyl)-imidazolidin-2-ylidene-amine or tautomer | 6 |
| 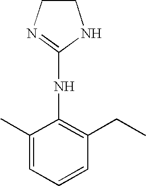 | (4,5-Dihydro-1H-imidazol-2-yl)-(2-ethyl-6-methyl-phenyl)-amine or tautomer | 7 |
| 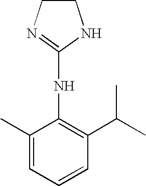 | (4,5-Dihydro-1H-imidazol-2-yl)-(2-isopropyl-6-methyl-phenyl)-amine or tautomer | 8 |
| 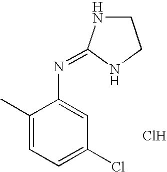 | (5-Chloro-2-methyl-phenyl)-imidazolidin-2-ylidene-amine or tautomer | 9 |
| 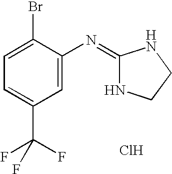 | (2-Bromo-5-trifluoromethyl-phenyl)-imidazolidin-2-ylidene-amine or tautomer | 10 |
| 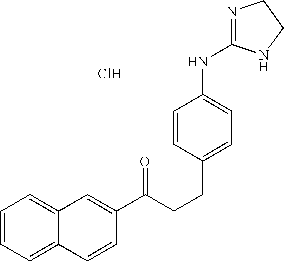 | 3-[4-(4,5-Dihydro-1H-imidazol-2-ylamino)-phenyl]-1-naphthalen-2-yl-propan-1-one ortautomer | 11 |
| 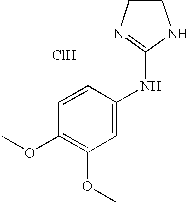 | (4,5-Dihydro-1H-imidazol-2-yl)-(3,4-dimethoxy-phenyl)-amine or tautomer | 12 |
| 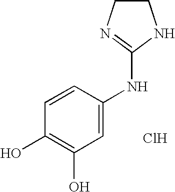 | 4-(4,5-Dihydro-1H-imidazol-2-ylamino)-benzene-1,2-diol or tautomer | 13 |
| 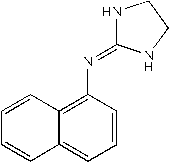 | Imidazolidin-2-ylidene-naphthalen-1-yl-amineor tautomer | 14 |
| 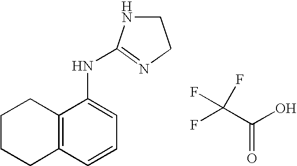 | (4,5-Dihydro-1H-imidazol-2-yl)-(5,6,7,8-tetrahydro-naphthalen-1-yl)-amine or tautomer | 15 |
| 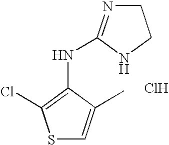 | (2-Chloro-4-methyl-thiophen-3-yl)-(4,5-dihydro-1H-imidazol-2-yl)-amine or tautomer | 16 |
| 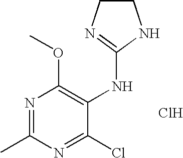 | (4-Chloro-6-methoxy-2-methyl-pyrimidin-5-yl)-(4,5-dihydro-1H-imidazol-2-yl)-amine ortautomer | 17 |
| 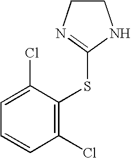 | 2-(2,6-Dichloro-phenylsulfanyl)-4,5-dihydro-1H-imidazole | 18 |

The compounds of formula I and their pharmaceutically acceptable addition salts possess valuable pharmacological properties. Specifically, the compounds of the present invention have a good affinity to the trace amine associated receptors (TAARs), especially TAAR1.

The compounds were investigated in accordance with the test given hereinafter.

### Materials and Methods

### Construction of TAAR Expression Plasmids and Stably Transfected Cell Lines

For the construction of expression plasmids the coding sequences of human, rat and mouse TAAR 1 were amplified from genomic DNA essentially as described by Lindemann et al. [14]. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC # CRL-1573) were cultured essentially as described Lindemann et al. (2005). For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hrs post transfection the culture medium was supplemented with 1 mg/ml G418 (Sigma, Buchs, Switzerland). After a culture period of about 10 d clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

### Membrane Preparation and Radioligand Binding

Cells at confluence were rinsed with ice-cold phosphate buffered saline without Ca²⁺ and Mg²⁺ containing 10 mM EDTA and pelleted by centrifugation at 1000 rpm for 5 min at 4° C. The pellet was then washed twice with ice-cold phosphate buffered saline and cell pellet was frozen immediately by immersion in liquid nitrogen and stored until use at −80° C. Cell pellet was then suspended in 20 ml HEPES—NaOH (20 mM), pH 7.4 containing 10 mM EDTA, and homogenized with a Polytron (PT 3000, Kinematica) at 10,000 rpm for 10 s. The homogenate was centrifuged at 48,000×g for 30 min at 4° C. and the pellet resuspended in 20 ml HEPES—NaOH (20 mM), pH 7.4 containing 0.1 mM EDTA (buffer A), and homogenized with a Polytron at 10,000 rpm for 10 s. The homogenate was then centrifuged at 48,000×g for 30 min at 4° C. and the pellet resuspended in 20 ml buffer A, and homogenized with a Polytron at 10,000 rpm for 10 s. Protein concentration was determined by the method of Pierce (Rockford, Ill.). The homogenate was then centrifuged at 48,000×g for 10 min at 4° C., resuspended in HEPES—NaOH (20 mM), pH 7.0 including MgCl₂ (10 mM) and CaCl₂ g protein per ml and (2 mM) (buffer B) at 200 homogenized with a Polytron at 10,000 rpm for 10 s.

Binding assay was performed at 4° C. in a final volume of 1 ml, and with an incubation time of 30 min. The radioligand [³H]-rac-2-(1,2,3,4-tetrahydro-1-naphthyl)-2-imidazoline was used at a concentration equal to the calculated K_{d} value of 60 nM to give a bound at around 0.1% of the total added radioligand concentration, and a specific binding which represented approximately 70-80% of the total binding. Non-specific binding was defined as the amount of [³H]-rac-2-(1,2,3,4-tetrahydro-1-naphthyl)-2-imidazoline bound in the presence of the appropriate unlabelled ligand (10 μM). Competing ligands were tested in a wide range of concentrations (10 pM-30 μM). The final dimethylsulphoxide concentration in the assay was 2%, and it did not affect radioligand binding. Each experiment was performed in duplicate. All incubations were terminated by rapid filtration through UniFilter-96 plates (Packard Instrument Company) and glass filter GF/C, pre-soaked for at least 2 h in polyethylenimine 0.3%, and using a Filtermate 96 Cell Harvester (Packard Instrument Company). The tubes and filters were then washed 3 times with 1 ml aliquots of cold buffer B. Filters were not dried and soaked in Ultima gold (45 μl/well, Packard Instrument Company) and bound radioactivity was counted by a TopCount Microplate Scintillation Counter (Packard Instrument Company).

The preferred compounds show a Ki value (μM) on mouse TAAR1 in the range of 0.026-0.500. Representative compounds are shown in the table below.

| | Ki (μM) | | |
|---|---|---|---|
| Example | mouse | Example | Ki |
| 2 | 0.149 | 11 | 0.121 |
| 3 | 0.026 | 14 | 0.036 |
| 6 | 0.501 | 15 | 0.039 |
| 7 | 0.169 | 16 | 0.294 |
| 8 | 0.476 | 18 | 0.030 |
| 9 | 0.225 | | |

The present invention also provides pharmaceutical compositions containing compounds of the invention, for example compounds of formula (I) and their pharmaceutically acceptable acid addition salts, and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compounds of the invention, in addition to one or more compounds of the invention, contain a pharmaceutically acceptable carrier. Suitable pharmaceutically acceptable carriers include pharmaceutically inert, inorganic and organic carriers. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The invention also provides a method for preparing compositions of the invention which comprises bringing one or more compounds of formula I and/or pharmaceutically acceptable acid addition salts thereof and, if desired, one or more other therapeutically valuable substances into a galenical administration form together with one or more therapeutically inert carriers.

The most preferred indications in accordance with the present invention are those, which include disorders of the central nervous system, for example the treatment or prevention of schizophrenia, cognitive impairment and Alzheimer's disease.

The dosage at which the compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

1. A method for treating a disorder selected from the group consisting of depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders which comprises administering to an individual a therapeutically effective amount of a compound of formula I 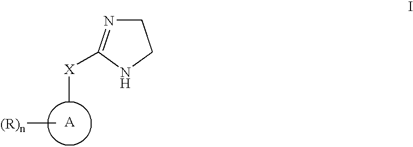
wherein
R is hydrogen, hydroxy, lower alkyl, lower alkoxy, halogen, lower alkyl substituted by halogen, or 4-(CH₂)₂C(O)-naphthyl;
X is —S— or —NH—;
A is aryl or hetaryl
aryl is an aromatic group selected from the group consisting of phenyl, naphthalen-1-yl, naphthalen-2-yl and 5,6,7,8-tetrahydronaphthalen-1-yl;
hetaryl is an aromatic group containing at least one N or S ring atom selected from the group consisting of thiophen-3-yl and pyrimidin-5-yl; and
n is 1, 2 or 3;
or a pharmaceutically active salt, racemic mixture, enantiomer, optical isomer or tautomeric form thereof.

2. The method of claim 1, wherein X is N, and aryl is phenyl.

3. The method of claim 2, wherein the compound is selected from the group consisting of
(4,5-dihydro-1H-imidazol-2-yl)-(2,6-dimethyl-phenyl)-amine or tautomer,
(2,6-diethyl-phenyl) -(4,5-dihydro-1H-imidazol-2-yl)-amine or tautomer,
(2,6-dibromo-phenyl)-imidazolidin-2-ylidene-amine or tautomer,
(4,5-dihydro-1H-imidazol-2-yl)-(2-ethyl-6-methyl-phenyl)-amine or tautomer,
(4,5-dihydro-1H-imidazol-2-yl)-(2-isopropyl-6-methyl-phenyl)-amine or tautomer,
(5-chloro-2-methyl-phenyl)-imidazolidin-2-ylidene-amine or tautomer and
3-[4-(4,5-dihydro-1H-imidazol-2-ylamino)-phenyl]-1-naphthalen-2-yl-propan-1-one or tautomer.

4. The method of claim 1, wherein X is N, and aryl/hetaryl is naphtha-1-yl, 5,6,7,8-tetrahydronaphthalen-1-yl or thiophen-3-yl

5. The method of claim 4, wherein the compound is selected from the group consisting of imidazolidin-2-ylidene-naphthalen-1-yl-amine or tautomer,
(4,5-dihydro-1H-imidazol-2-yl)-(5,6,7,8-tetrahydro-naphthalen-1-yl)-amine trifluoro-acetate or tautomer and
(2-chloro-4-methyl-thiophen-3-yl)-(4,5-dihydro-1H-imidazol-2-yl)-amine or tautomer.

6. The method of claim 1, wherein X is S and aryl is phenyl.

7. The method of claim 6, wherein the compound is
2-(2,6-dichloro-phenylsulfanyl)-4,5-dihydro-1H-imidazole.

8. The method of claim 1, wherein the disorder is schizophrenia.

9. The method of claim 1, wherein the disorder is Alzheimer's disease.

10. The method of claim 1, wherein the disorder is cognitive impairment.

11. A method for preparation of compounds of formula I 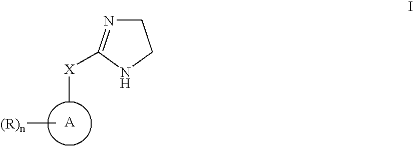
wherein
R is hydrogen, hydroxy, lower alkyl, lower alkoxy, halogen, lower alkyl substituted by halogen, or 4-(CH₂)₂C(O)-naphthyl;
X is —S— or —NH—;
A is aryl or hetaryl
aryl is an aromatic group selected from the group consisting of phenyl, naphthalen-1-yl, naphthalen-2-yl and 5,6,7,8-tetrahydronaphthalen-1-yl;
hetaryl is an aromatic group containing at least one N or S ring atom selected from the group consisting of thiophen-3-yl and pyrimidin-5-yl; and
n is 1, 2 or 3;
or a pharmaceutically active salt, racemic mixture, enantiomer, optical isomer or tautomeric form thereof selected from the group consisting of
a) reacting a compound of formula 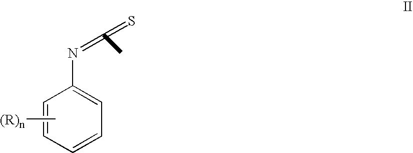
with ethylenediamine of formula
H₂NCH₂CH₂NH₂ III
to obtain a compound of formula 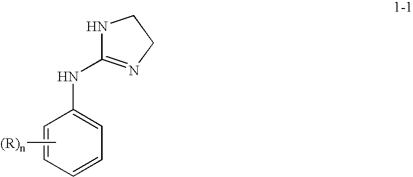
and
b) reacting a compound of formula 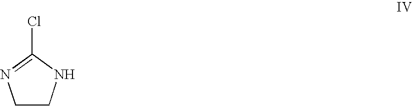
with a compound of formula 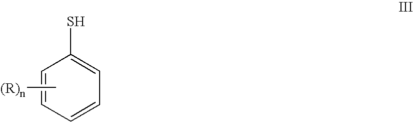
to obtain a compound of formula

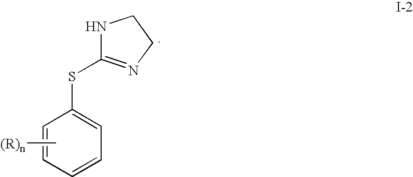

12. A pharmaceutical composition comprising a compound of formula I 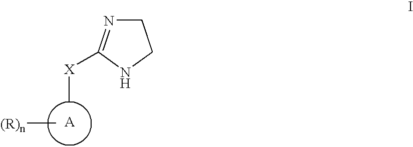
wherein
R is hydrogen, hydroxy, lower alkyl, lower alkoxy, halogen, lower alkyl substituted by halogen, or 4-(CH₂)₂C(O)-naphthyl;
X is —S— or —NH—;
A is aryl or hetaryl
aryl is an aromatic group selected from the group consisting of phenyl, naphthalen-1-yl, naphthalen-2-yl and 5,6,7,8-tetrahydronaphthalen-1-yl;
hetaryl is an aromatic group containing at least one N or S ring atom selected from the group consisting of thiophen-3-yl and pyrimidin-5-yl; and
n is 1, 2 or 3;
or a pharmaceutically active salt, racemic mixture, enantiomer, optical isomer or tautomeric form thereof and a pharmaceutically acceptable carrier.
